(19) United States
(12) Patent Application Publication
(10) Pub. No.: US 2004/0054279 A1
(43) Pub. Date: Mar. 18, 2004

(54) Catheter steering apparatus and method

(76) Inventor: Peter Hanley, Gloucestershire (GB)

Correspondence Address: STAAS & HALSEY LLP, SUITE 700, 1201 NEW YORK AVENUE, N.W., WASHINGTON, DC, 20005, US

(21) Appl. No.: 10/416,576

(22) PCT Filed: Nov. 15, 2001
(86) PCT No.: PCT/GB01/05026

(30) Foreign Application Priority Data
Nov. 29, 2000 (GB) 0029158.3

(51) Int. Cl. A61B 5/05

(52) U.S. Cl. 600/424

(57) ABSTRACT

A catheter steering assembly (1) is provided for attachment to a catheter (7) so as to enable the catheter to be steered within an applied magnetic field. The catheter steering assembly (1) comprises a magnetic element (3) formed from a material having a controllable magnetisation direction and at least one control element (4a, 4b, 4c) arranged to interact with the magnet element (3) so as to control the magnetisation direction of the magnetic element (3). The material of the magnetic element is arranged such that the magnetisation direction is maintained for a period after the interaction, whereby the catheter steering assembly (1) may be steered by the interaction between the magnetic field of the magnetic element (3) and the applied magnetic field.

The present invention relates to an apparatus and method for steering a catheter.

Catheterisation is a surgical procedure which is regarded as being minimally invasive and therefore less traumatic to the patient than more conventional open surgery techniques. Typical applications of such procedures include the repair of aneurisms and the removal of obstructions within blood vessels such as thromboses. For example the occlusion of coronary arteries can be effectively treated using catheterisation procedures and this is now a relatively common practice.

Traditionally, guidance of the catheter in a body cavity or lumen is achieved by the surgeon manually applying a torque to the bent tip of a catheter or guide wire along with providing an axial force to push the catheter forward. The progress of the catheter is monitored during this process using imaging techniques such as ultrasound or X-ray fluoroscopy. However, ultrasound suffers from poor spatial resolution and provides images that are difficult to interpret, whereas X-ray fluoroscopy has related safety implications due to the repeated use of radiation. More recently there has been increased interest in the use of magnetic resonance imaging (MRI) for monitoring this procedure.

The traditional method of steering a catheter using a combination of torque and axial force has associated problems in that there is a risk of trauma within the patient particularly when navigating the catheter along a tortuous path. Surgeons can also suffer from physical fatigue during long procedures.

A number of magnetic steering methods have been proposed for steering catheters, These involve the insertion of a permanent magnet device in to the body either in addition to or as part of the catheter. An external magnetic field is then provided and by controlling the applied magnetic field or magnetic field gradients, the orientation of the magnetic device within the body is affected, resulting in the steering of the catheter. An example of such a catheter is described in WO99/40957.

Stereotaxis Inc have proposed various apparatus and methods for applying and manipulating such an applied magnetic field, for example using superconducting coils. Examples of such systems are disclosed in WO99/23946 and WO99/11189.

However, in order to produce the desired magnetic field or magnetic field gradient at the position of the catheter, a high degree of control is required over the applied magnetic fields. In addition very precise positioning of the patient with respect to these fields is also necessary.

As strong magnetic fields are required to produce a sufficient resultant force upon the catheter, the apparatus required to generate such fields is often bulky and in particular does not lend itself to simultaneous imaging methods for monitoring the procedure. The desire to use magnetic resonance imaging provides further complications in that the presence of magnetic fields or magnetic field gradients in addition to those provided by the MRI apparatus can cause significant distortion of the resultant images.

In “Integrating X-Ray Angiography and MRI for Endovascular Interventions”, T.P.L. Roberts et al., Medica Mundi (Philips Medical Systems), 44/3, November 2000, a brief description is given of a device in which one or a series of coils are wound around the tip of a catheter. A magnetic moment is generated by passing an electrical current through the coil(s) to effect steering by interaction with an external magnetic field.

In accordance with a first aspect of the present invention we provide a catheter steering assembly for attachment to a catheter so as to enable the catheter to be steered within an applied magnetic field, the catheter steering assembly comprising:

a magnetic element formed from a material having a controllable magnetisation direction; and

at least one control element arranged to interact with the magnetic element so as to control the magnetisation direction of the magnetic element, the material of the magnetic element being such that the magnetisation direction is maintained for a period after the interaction whereby the catheter steering assembly may be steered by the interaction between the magnetic field of the magnetic element and the applied magnetic field.

We have realised that a number of the problems associated with prior art methods can be addressed by providing the magnetic element with a controllable magnetisation direction. This can then be used to steer the catheter. Unlike in prior systems, where the external field is manipulated with respect to a fixed permanent magnet, this new approach controls the magnetisation direction of the magnetic element itself and may be used in association with a uniform applied magnetic field. The control of this assembly is therefore achieved at the position where steering is effected rather than remotely via a combination of applied magnetic fields.

Typically the applied external magnetic field can therefore be greatly simplified allowing a corresponding simplification in the apparatus required to produce such a field. This provides benefits when using the apparatus in association with other imaging apparatus.

An important advantage is provided by using a magnetic element in which the magnetisation direction persists for a period following the interaction. For example, if the magnetic field within the catheter steering assembly is generated and maintained only using coils carrying an electrical current (as in the Roberts et al paper), then typically the strength of the magnetic field required causes a corresponding large power dissipation in these coils. This is of course a significant problem when the catheter is within the body of a subject and extensive damage to the body tissues may occur.

The catheter steering assembly may be provided as an individual entity for attachment to the catheter or alternatively may be provided as part of the catheter itself.

Preferably a suitable material for magnetisation will be chosen such that the magnetisation magnitude and direction relative to the catheter tip are retained in the externally applied magnetic field, until caused to change by the control element. An example of such a material would be a hard, isotropic ferrite, which could retain a magnetisation of 0.2 Tesla in an applied external field of 250 kA/m at a temperature of 37° C. A variety of these “hard” magnetic materials are available.

Magnetisation of the magnetic element may be conveniently provided using one or more control elements as electromagnets in the form of coils. By providing an electric signal such as a pulse within such a coil, a sufficient magnetic field may be generated to cause the magnetic element to adopt a magnetisation direction in accordance with the applied field.

When the control element(s) of the present invention take the form of a number of electrical coils, preferably these coils are used primarily in establishing the magnetic field direction in the magnetic element. Typically the current is then terminated and the magnetic element retains the magnetic field so produced for interaction with the (external) applied magnetic field. This period of interaction with the applied field is typically of the order of seconds or minutes. Therefore the power dissipation is much reduced with respect to a system in which the magnetic field is maintained throughout this period using an electrical current in the coils.

The control element (s) will also preferably be adapted to control the magnitude of the magnetisation such that the magnetic element may be substantially demagnetised if required. This is particularly advantageous when using the assembly with MRI imaging as very little distortion of the image will result.

Preferably, one or more control elements may be provided to produce a magnetisation direction lying in an arbitrary direction. One way of achieving this using electrically conducting coils is to arrange each coil to enclose the magnetic element with the coils being arranged in a substantially orthogonal manner with respect to one another. The coils will preferably take the form of complete rings fully encircling the magnetic element although control elements that only partially enclose the magnetic element could also be used.

Typically the magnetic element will be provided as a substantially spherical magnetisable body having isotropic magnetisation properties.

In general the magnetic element and/or the one or more control elements will be supported by a support structure such as a housing, the support structure being provided with a suitable connection to the catheter. Alternatively, the support structure may itself form the tip of a catheter.

In general, the catheter steering assembly will be used for steering the catheter in addition to an separate propulsive method, for example using a guide wire. However, the magnetisation of the magnetic element could be used to provide forward motion of the catheter.

In accordance with a second aspect of the present invention, we provide:

a catheter steering assembly according to the first aspect of the invention; and

a catheter attached to the catheter steering assembly.

In accordance with a third aspect of the present invention, we provide a catheter steering system comprising:

a catheter steering assembly according to the first aspect of the invention;

a catheter attached to the catheter steering assembly; and,

a control system for controlling the control element of the catheter steering assembly.

In general the control system of the catheter steering system will comprise a number of control lines. These provide electrical signals such as electric pulses to the control element(s) so as to affect the magnetisation direction of the magnetic element. The electrical signals will generally by provided by a signal generator. The system may further comprise a computer having a corresponding processor to interpret the instructions of a surgeon and control the signal generator accordingly.

The catheter steering system may further comprise a magnetic device for generating the external applied magnetic field. In general the applied magnetic field will be arranged to be substantially uniform. This magnetic field may be provided by a magnetic resonance imaging device for convenient use as part of the catheter steering system and also as an imaging device. Preferably in this case, the control system will be capable of substantially demagnetising the magnetic element so as to allow a non-distorted image to be produced of the catheter region.

In accordance with a fourth aspect of the present invention we provide a method of operating a catheter steering system according to the third aspect of the invention, the method comprising:

inserting the catheter steering assembly attached to a catheter to a first location; and

applying a magnetic field;

causing the catheter to move from the first location along a path; and,

operating the control system to control the magnetisation of the magnetic element such that the catheter is steered as it is caused to move.

An example of a catheter steering method and system will now be described with reference to the accompanying drawings, in which:-

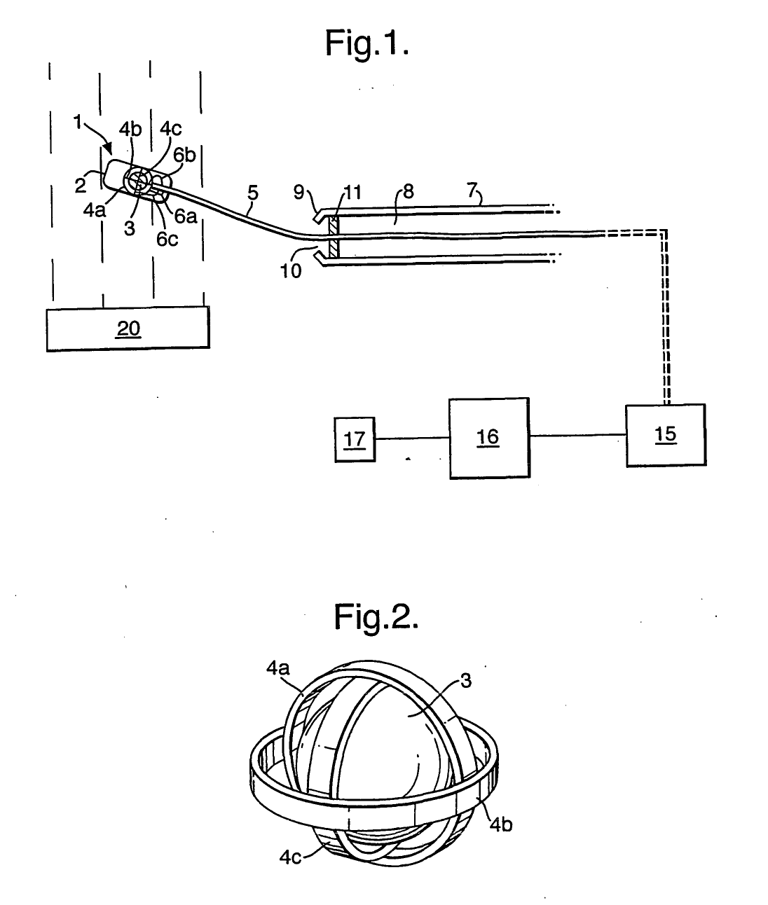

FIG. 1 is a schematic representation of a catheter steering system according to the example;

FIG. 2 is an illustration of a catheter steering assembly according to the example;

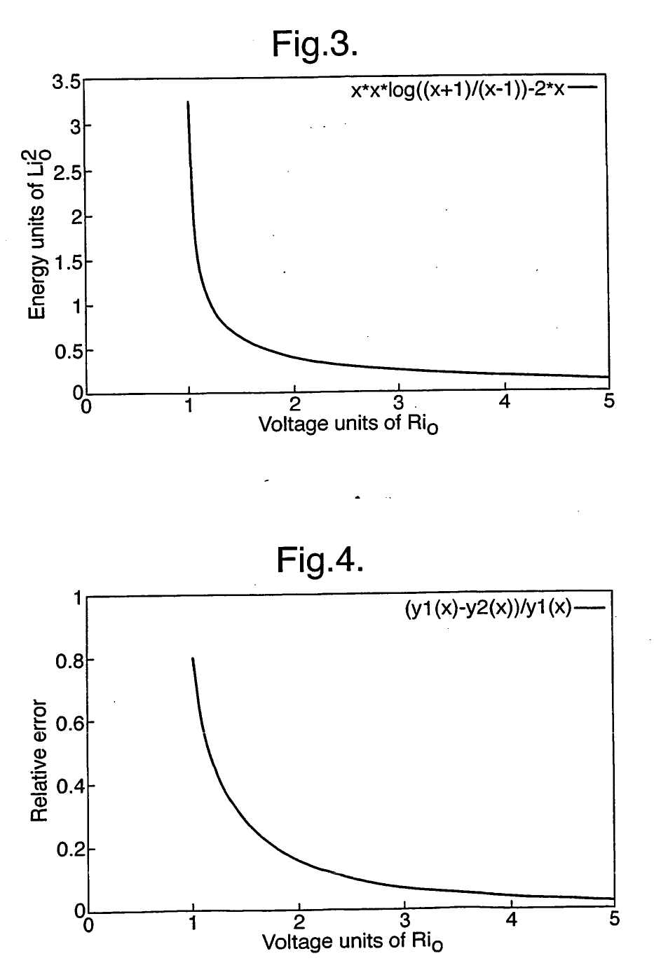

FIG. 3 is a graph illustrating the energy dissipation function of a coil according to the example;

FIG. 4 is a graph illustrating the error in the energy dissipation function;

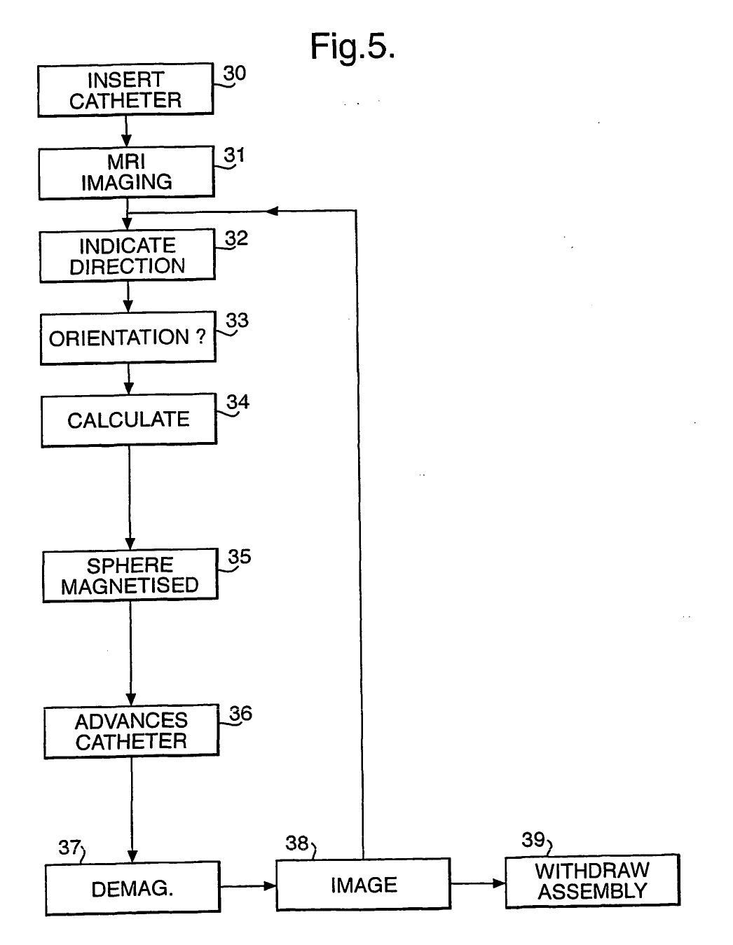

FIG. 5 is a flow diagram illustrating the method of operating the catheter steering system according to the example;

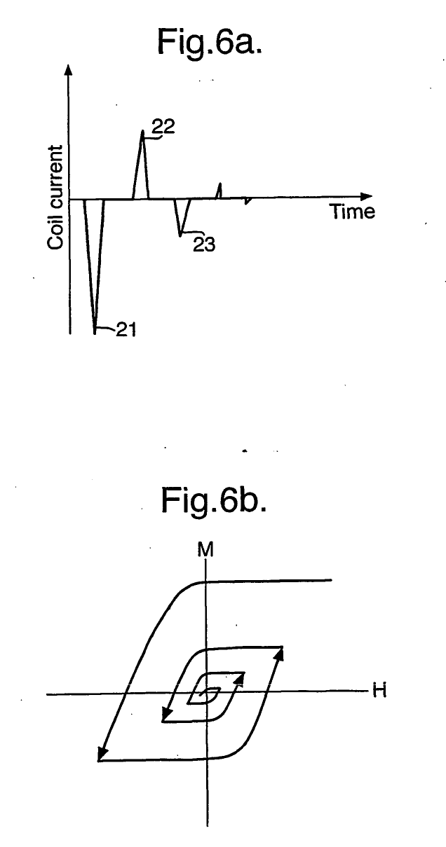

FIG. 6a is an illustration of the electrical signals applied to the catheter steering assembly in order to demagnetise the magnetic element; and,

FIG. 6b shows the corresponding reduction in the magnetisation.

FIG. 1 shows a catheter steering assembly generally indicated at 1, the catheter steering assembly comprising a housing 2 within which is enclosed a sphere 3 of hard magnetic material such as ferrite, the sphere being enclosed by three orthogonal electric coils 4a,4b,4c.

A guide wire 5 is connected to the housing 2, the guide wire containing electrical lines 6a,6b,6c for supplying electric signals to the electrical coils 4a,4b,4c respectively. The guide wire 5 passes through a catheter generally indicated at 7, the catheter having an elongate body and a central bore 8 through which the guide wire passes.

At the end of the catheter body closest to the catheter steering assembly 1, an annular lip 9 is provided so as to narrow the diameter of the bore 8 to form an opening 10. At a predetermined distance along the guide wire from the catheter steering assembly 1, a disk 11 is attached to the guide wire 5, the radius of the disk being arranged to be just less than that of the internal diameter of the catheter 7 and yet larger than the diameter of the opening 10. During use, the attachment of the disk to the guide wire 5, prevents the catheter steering assembly 1 from separating from the catheter 7 by more than a predetermined distance. This distance can be arranged according to the use of the catheter in question.

The catheter guide assembly 1 and catheter 7, along with the guide wire 5, are formed from suitable materials to be used within the body of a living subject such as the human body. The guide wire 5 is lead out of the body and is adapted for manipulation by a surgeon. In this example the guide wire has sufficient stiffness to allow the catheter steering assembly 1 and catheter 7 to be moved through body cavities or lumens by applying a sufficient axial force to the guide wire 5.

The electrical lines 6a,6b,6c are attached to an external signal generator 15 which is adapted to provide electrical signals to the respective electrical lines 6a,6b,6c. The signal generator 15 is controlled by a computer 16 having a processor operating control software. An appropriate input device 17 such as a keyboard or joystick allows the surgeon to control the electrical signals being passed to the catheter steering assembly 1 using the computer 16.

A magnet, schematically represented at 20 is positioned so as to apply a magnetic field with which the catheter steering assembly 1 may interact. Generally, the magnet 20 will comprise a number of electromagnets, suitably arranged with respect to the body of the subject. The control of the magnet 20 will generally be achieved using a processor, such as the processor contained within the computer 16.

FIG. 2 shows the catheter steering assembly 1 in more detail, with the housing 2 removed. The ferrite sphere 3 is encircled by the three electric coils 4a,4b,4c. Each of these coils comprises a number of turns of high conductivity electrical wire, the coils being electrically connected to the electrical signal generator 15 using the corresponding electrical lines 6a,6b,6c positioned along the guide wire 5.

As indicated in FIG. 2, the three coils are arranged about the centre of the sphere 3 along mutually orthogonal axes. If sufficiently isotropic ferrite is used for the sphere 3, then the arrangement of the coils 4a,4b,4c in this manner allows the generation of a magnetic field within the ferrite in an arbitrary direction by superposition of the fields generated by each coil individually. This may be achieved by applying one or more suitable current pulses to one or more of the coils such that the combined magnetic field generated by the current in the coils is greater than the coercive force required to move the magnetic domains within the material.

In this manner, not only can the direction of the magnetisation be changed at will, the magnitude and polarity of this magnetisation can also be controlled. An additional benefit is that a series of electrical pulses can provide the facility of demagnetising the ferrite for subsequent MRI imaging.

An analysis of the physics relating to the catheter steering assembly 1 of this example, will now be described. The couple on a magnetic dipole is

\vec{Γ}=\vec{m} [symbol] \vec{H}

● For a coil

\vec{m}=μ₀·I·\vec{A}

where I is the number of ampere-turns and |A| is the area of the coil with the ve [symbol] \vec{A} in the direction perpendicular to plane of the coil. For a circular loop m=μ₀·I·π·a²

● For a permanent magnet

\vec{m}=∫\vec{M}dV

where M is the magnetisation per unit volume.

For a uniformly magnetised sphe

[symbol]

$$\vec{m} = \frac{4}{8} \cdot I \cdot π \cdot {a}^{3} \cdot \vec{M}$$

Hence for a coil to have the same effect as a sphere of magnetic material

$${μ}_{0} \cdot I = \frac{4}{3} \cdot a \cdot M$$

For M=1 Tesla (eg. NdFeB), and a≈1 mm, I≃1000 A-turns.

To switch a pernanent magnet we need to apply a pulse of field stronger than the coercive force. (H [symbol] 300,000 Am⁻¹ for ferrite). Also, the background field should not be greater than H_{c}. Using

$$H = \frac{I}{2 a}$$

we again find that we need about 1000A-turns. Clearly, the power dissipation of 1000 A-turns in a small volume presents a problem. In the “switched pernani [symbol] magnet” implementation, the switching pulse can be short so as to reduce the total energy.

If the coil is characterised by an inductance L and a resistance R, then applying a voltage V across it cau [symbol] the current to rise according to

$$i = \frac{v}{R} ( 1 - exp ( - \frac{t}{L / R} ) )$$

The time taken to reach the desired current, i₀, is

$${t}_{1} = - \frac{L}{R} ln ( 1 - \frac{R}{V} {i}_{0} )$$

and the energy dissipated in this time is

$${\int }_{0}^{{t}_{1}} {i}^{2} R ⅆ t .$$

The magnetisation having been switched, we now wish to de-energise the coil as quickly as possible so [symbol] apply a similar voltage in the reverse direction, so that the current falls as

$$i = ( {i}_{0} + \frac{V}{R} ) exp ( - \frac{t}{L / R} ) - \frac{V}{R}$$

the time taken to reach zero is

$${t}_{0} = \frac{L}{R} ln ( 1 + \frac{R}{V} {i}_{0} )$$

and the energy dissipated in this part of the cycle is

$${\int }_{0}^{{t}_{0}} {i}^{2} R ⅆ t .$$

The total energy dissipated in the cycle is

$$E = L \frac{{V}^{2}}{{R}^{2}} ln ( \frac{V + R \cdot {i}_{0}}{V - R \cdot {i}_{0}} ) - 2 \cdot L \cdot {i}_{0} \frac{V}{R}$$

Writing the energy in units of L·i²₀ and voltage in units of R·i₀ so that

$$\hat{E} = \frac{E}{L \cdot {i}_{0}^{2}}$$

and

$$x = \frac{V}{R \cdot {i}_{0}}$$

$$\hat{E} = {x}^{2} \cdot ln ( \frac{x + 1}{x - 1} ) - 2 \cdot x$$

This function is plotted in FIG. 3 To minimise the dissipation, the voltage should be as high as possible, preferably >3·i₀·R If this is so, then the current rise is approximately linear

$$i = \frac{V}{L} \cdot t$$

and the total dissipation per cycle is

$$E ≃ \frac{2 \cdot L \cdot R}{3 \cdot V} {i}_{0}^{3} = \frac{2}{3} L \cdot {i}_{0}^{2} \frac{{i}_{0} \cdot R}{V} .$$

The relative difference between this and the exact expression is shown in FIG. 4 Let the coil with I ampere-turns have N turns and current i₀ so that I=N·i₀. If an “average” winding radi [symbol] is a, the length of wire is [symbol] =·2·π·a·N and its volume is Vol= [symbol] ·A where is the cross-sectional area of the wire. Assuming adiabatic conditions, the current pulse causes a temperature rise of

$$δΘ = \frac{E}{s \cdot Vol} ≃ \frac{2 \cdot L \cdot R}{3 \cdot V \cdot s \cdot Vol} {i}_{0}^{3} = \frac{2 \cdot L \cdot R}{s \cdot V \cdot s \cdot Vol} {( \frac{2 \cdot a \cdot {H}_{c}}{N} )}^{3}$$

where s is the volume specific heat. We now need to derive expressions for the inductance and resistance.

$$\begin{matrix}L ≃ μ \cdot {μ}_{0} \cdot \frac{π}{2} \cdot {N}^{2} \cdot a \\ R = ρ \cdot \frac{t}{A} \end{matrix}$$

Vol= [symbol] ·A

where A is the cross-sectional area of the wire. We therefore find

$$δΘ = \frac{8}{3} \frac{μ \cdot {μ}_{0} \cdot π \cdot ρ \cdot {a}^{4} \cdot {H}_{c}^{3}}{{A}^{2} \cdot N \cdot V \cdot e}$$

We must also satisfy the condition

$$V \geq 3 \cdot R \cdot {i}_{0} = \frac{δ π \cdot ρ \cdot a \cdot I}{A} A \geq \frac{12 \cdot π \cdot ρ \cdot {a}^{2} \cdot {H}_{c}}{V}$$

Before attempting to evaluate these expressions, we need to consider the meaning of the relative permeability μ. This appears in the expression for the inductance, which determines how quickly the coil can be charged and consequently bow long it spends with current in it. The BH curve of a typical permanent magnet shoi [symbol] that μ varies considerably as the H field is varied from zero to H_{c}. An average value can be arrived at fro [symbol] consideration of the energy change:

$$\frac{1}{2} L \cdot {i}_{0}^{2} ≃ {B}_{rem} \cdot {H}_{c} \cdot \frac{4 \cdot π \cdot {a}^{2}}{3}$$

whence

$${μ}_{av} = \frac{4 \cdot {B}_{rem}}{3 \cdot {μ}_{0} \cdot {H}_{c}}$$

It must also be noted that if the number of turns is not large, then the lead resistance must also be taken in [symbol] account. In this case, the expression for δθ is unchanged, but the voltage should now satisfy the condition

$$V \geq K \cdot \frac{ρ \cdot ( 2 π \cdot a \cdot N + {l}_{leads} )}{A} \frac{2 \cdot a \cdot {H}_{c}}{N}$$

where K≃3

The results of a spread sheet calculation using this are shown below: All units in metres, kilogramne, seconds, Amperes

| rho | Brem | Hc | a | deltaTheta | | muO | | s | Lead length |
|---|---|---|---|---|---|---|---|---|---|
| (ohm-m) | (Tesla) | (A/m) | (m) | (K) | mu | (H/m) | K | (J/m³¹ 3/K) | (m) |
| 2.00E−08 | 0.60 | 3.00E+05 | 2.00E−03 | 100.00 | 3.18 | 1.26E−06 | 3.00 | 3.38E+ 06 | 1.00 |

| N | A | V | i | L | wire diam | R | t |
|---|---|---|---|---|---|---|---|
| 1 | 1.61E−07 | 4.53E+02 | 1200.00 | 1.26E−08 | 2.26E−04 | 1.26E−01 | 4.02E−08 |
| 2 | 6.29E−07 | 5.87E+01 | 600.00 | 5.03E−08 | 4.47E−04 | 3.26E−02 | 6.25E−07 |
| 5 | 3.65E−06 | 4.19E+00 | 240.00 | 3.14E−07 | 1.08E−03 | 5.82E−03 | 2.19E−05 |
| 10 | 1.30E−05 | 6.22E+01 | 120.00 | 1.26E−06 | 2.04E−03 | 1.73E−03 | 2.95E−04 |
| 20 | 4.22E−05 | 1.07E+01 | 60.00 | 5.03E−06 | 3.66E−03 | 5.93E−04 | 3.44E−03 |
| 50 | 1.56E−04 | 1.51E+02 | 24.00 | 3.14E−05 | 7.04E−03 | 2.09E−04 | 6.09E−02 |
| 100 | 3.24E−04 | 5.01E+03 | 12.00 | 1.26E−04 | 1.02E−02 | 1.39E−04 | 3.66E−01 |
| 200 | 5.35E−04 | 2.36E+03 | 6.00 | 5.03E−04 | 1.31E−02 | 1.31E−04 | 1.55E−00 |
| 500 | 7.78E−04 | 1.35E+03 | 2.40 | 3.14E−03 | 1.57E−02 | 1.87E−04 | 6.81E−00 |
| 1000 | 8.97E−04 | 1.09E+03 | 1.20 | 1.26E−02 | 1.69E−02 | 3.02E−04 | 1.68E−01 |
| 2000 | 9.67E−04 | 9.73E+04 | 0.60 | 5.03E−02 | 1.75E−02 | 5.40E−04 | 3.77E−01 |

As will be appreciated from the above, were the ferrite sphere 3 removed from the catheter steering assembly 1 of the present example, the magnetisation would not persist after the removal of the current from the coil.

The magnetisation of the ferrite can be produced by a brief pulse of current through the coil. The associated power dissipation for an appropriate magnetisation pulse in the coil of the above example is about 1.8 mJ. In contrast, for a steering operation requiring the magnetisation to persist for a minute, to produce a similar dipole moment in a coil of comparable size would require a continuous dissipation of about 100 watts (and therefore 6000 J in total). It will be appreciated that the dissipation of 6000 J of energy within a small area of the body of a subject would be extremely difficult to manage and would be likely to cause much damage to the subject's tissues.

A method of operating the combined catheter steering assembly and associated catheter will now be described in association with FIGS. 5 and 6. The method is performed in association with a continuous external applied magnetic field provided by the magnet(s) 20. The magnitude of this magnetisation is less than the coercive force of the sphere material.

At step 30 in FIG. 5 the catheter steering assembly 1 and catheter 7 are inserted into the body at an appropriate position, for example in a major blood vessel. An axial force is applied to the guide wire so as to urge the catheter and catheter steering assembly to a point where intricate steering may be required. During this process, the disk 11 attached to the guide wire 5, impacts against the lip 9 so as to push the catheter along the respective blood vessel.

During this initial locating of the catheter 7, the ferrite sphere 3 is provided in a non-magnetised state so as to allow the progress of the procedure to be periodically monitored using an MRI imager at step 31. The orientation of the catheter steering assembly 1 can be determined from MRI (or other imaging modalities) by using markers on the catheter steering assembly 1.

The imaging process allows the surgeon to monitor the position of the catheter steering assembly 1 with respect to the blood vessel in question. When steering of the catheter 7 is required, for example at a blood vessel junction, the surgeon indicates to the computer 16 his desire to steer the catheter steering assembly 1 in a particular direction. Using the input device 17, he may then indicate the present orientation of the catheter steering device 1, with respect to the applied magnetic field on the magnet 20. Alternatively, the computer may track the orientation of the device by analysing the respective MRI images or using other sensing means such as monitoring the current produced in the respective coils due to motion of the catheter steering assembly 1.

At step 32, the surgeon indicates to the computer the direction in which he wishes the catheter steering assembly 1 to move. The orientation of the catheter steering assembly is then deduced at step 33 and, at step 34 by knowing the orientation of the applied magnetic field and the orientation of the catheter steering assembly 1, the computer calculates the required orientation of the magnetic field of the sphere 3 so as to cause the catheter steering assembly 1 to move in the desired direction. Typical parameters in such calculations include the magnitude, polarity and direction of the magnetic field to be generated, along with the corresponding magnitude of the electrical pulses to be supplied to the three electric coils 4a to 4c as appropriate.

The computer 16 then instructs the electrical signal generator 15 to send one or more pulses to the coils 4a,4b,4c at step 35. The electrical pulses within each coil generate an associated magnetic field passing through the sphere 3, the magnetic field being of sufficient magnitude to overcome the coercive force required to move the magnetic domains within the sphere. The superposition of the magnetic fields generated by each coil produces a corresponding magnetic field within the sphere 3 which is retained after the electrical pulses have been applied.

The surgeon then advances the catheter steering assembly 1 manually with the guide wire at step 36 during which the catheter steering assembly 1 is steered by the magnetic field interaction. A further imaging process is then performed in order to confirm that the catheter has moved in the desired direction. However, if an MRI scanner is used, the magnetisation of the ferrite may prevent a sufficiently clear and undistorted image from being obtained. Therefore a demagnetisation process is performed at step 37 prior to imaging.

As shown in FIG. 6a, this involves the repeated delivery of electrical signal pulses 21,22,23 and so on, to the appropriate coils 4a,4b,4c with alternating polarity and decreasing magnitude. This sequence ensures that the magnetisation of the ferrite sphere 3 is reduced to a low level so as to allow MRI imaging to be performed, as shown in FIG. 6b. Following the demagnetisation conventional MRI imaging is performed at step 38.

Steps 32 to 38 may be repeated a number of times so as to allow the catheter to be guided along the desired path within the particular blood vessel or lumen in question.

Once correctly positioned within the body, the catheter steering assembly 1 may be withdrawn through the catheter and removed from the body by pulling upon the guide wire 5 (step 39). The width of the catheter steering assembly 1 in this case is therefore less than the diameter of the opening 10 such that following withdrawal at step 39, the catheter remains in position within the body.

In some cases a catheter steering assembly 1 may be permanently connected to the catheter for example using a flexible joint at the tip of the catheter. This may be used particularly where only temporary catheterisation is required.

Although the above example has been described in association with a uniform magnetic field, the catheter steering assembly according to the present invention may be used in association with a non-uniform applied magnetic field or magnetic field gradient.

1. A catheter steering assembly (1) for attachment to a catheter (7) so as to enable the catheter (7) to be steered within an applied magnetic field, the catheter steering assembly (1) comprising:
a magnetic element (3) formed from a material having a controllable magnetisation direction; and
at least one control element (4a,4b,4c) arranged to interact with the magnetic element (3) so as to control the magnetisation direction of the magnetic element (3), the material of the magnetic element (3) being such that the magnetisation direction is maintained for a period after the interaction whereby the catheter steering assembly (1) may be steered by the interaction between the magnetic field of the magnetic element (3) and the applied magnetic field.

2. An assembly according to claim 1, wherein the control element (4a,4b,4c) is adapted to control the magnitude of the magnetic element magnetisation.

3. An assembly according to claim 1 or claim 2, wherein the or each control element (4a,4b,4c) at least partially encloses the magnetic element.

4. An assembly according to any of claims 1 to 3, wherein the or each control element (4a,4b,4c) is an electrically conducting coil.

5. An assembly according to any of claims 1 to 4, further comprising two additional control elements, wherein each control element (4a,4b,4c) comprises an electrically conducting coil at least partially enclosing the magnetic element and wherein the control elements are arranged in a substantially orthogonal manner with respect to one another.

6. An assembly according to any of the preceding claims, wherein the magnetic element (3) is substantially spherical.

7. An asswmbly according to any of the preceding claims, wherein the material of the magnetic element (3) is a ferrite.

8. An assembly according to any of claims 1 to 7, further comprising a support structure for supporting at least one of the magnetic element or the control element, and wherein the support structure is adapted to be connected to the catheter.

9. An assembly according to claim 8, wherein the assembly comprises a tip of a catheter (7).

10. An assembly according to any of the preceding claims, wherein the magnetisation direction is permanently retained until caused to change by the control element (4a,4b,4c).

11. An assembly according to any of the preceding claims, wherein the magnetisation magnitude is permanently retained until caused to change by the control element (4a,4b,4c).

12. In combination:
a catheter steering assembly (1) according to any of claims 1 to 11; and
a catheter (7) attached to the catheter steering assembly.

13. A catheter steering system comprising:
a catheter steering assembly (1) according to any of claims 1 to 11;
a catheter (7) attached to the catheter steering assembly; and
a control system for controlling the control elements (4a,4b,4c) of the catheter steering assembly.

14. A catheter steering system (1) according to claim 13, wherein the control system is adapted to supply electric signals to the control elements of the catheter steering assembly in order to control the magnetisation of magnetic element.

15. A catheter steering system according to claim 4, wherein the control system comprises an electrical signal generator (15).

16. A catheter steering system according to claim 15, wherein the control system further comprises a computer (16) arranged to control the electrical signal generator (15).

17. A catheter steering system according to any of claims 13 to claim 16, further comprising a magnetic device for generating the applied magnetic field.

18. A catheter steering system according to any of claims 13 to claim 17, wherein the applied magnetic field is substantially uniform.

19. A method of operating a catheter steering system according to any of claims 13 to 18, the method comprising:
inserting the catheter steering assembly (1) attached to a catheter (7) to a first location; and
applying a magnetic field;
causing the catheter (7) to move from the first location along a path; and,
operating the control system to control the magnetisation of the magnetic element (3) such that the catheter (7) is steered as it is caused to move.

20. A method according to claim 19, further comprising, operating the control system to cause the magnetisation of the magnetic element (3) to reduce from a relatively high level of magnitude for use in steering, to a relatively low level of magnitude; and performing a magnetic resonance imaging (MRI) process.
